(19) United States
(12) Patent Application Publication
(10) Pub. No.: US 2006/0211936 A1
(43) Pub. Date: Sep. 21, 2006

(54) Flexible biomonitor

(75) Inventors: Ying-Chiang Hu, Bade City (TW); Yi-Tay Chiou, Kaohsiung City (TW); Chun-Hsun Chu, Tainan City (TW); Bor-Chen Tsai, Taipei City (TW)

Correspondence Address: BRUCE H. TROXELL, SUITE 1404, 5205 LEESBURG PIKE, FALLS CHURCH, VA, 22041, US

(73) Assignee: Industrial Technology Research Institute

(21) Appl. No.: 11/132,216

(22) Filed: May 19, 2005

(30) Foreign Application Priority Data
Mar. 21, 2005 (TW) 94108277

(51) Int. Cl. A61B 5/04; A61B 5/02; A61B 5/00

(52) U.S. Cl. 600/386; 128/903; 600/500; 600/549

(57) ABSTRACT

A flexible biomonitor comprises a flexible substrate, a circuit apparatus, a plurality of gauges, a RFID sensing chip, a micro-antenna, and a power supply. The flexible substrate has a plurality of through holes formed thereon. The circuit apparatus is electrically connected with the gauges via the through holes to sense and treat a physiological signal. The micro-antenna is electrically connected with the circuit apparatus to transmit this physiological signal. The power supply is designed to provide electric power. Thereupon the flexible biomonitor can be plastered on the skin where the human body needs to be monitored to achieve the purposes of reducing occupied area and providing comfortable wear. Besides, it is capable of remote real-time monitoring this signal to achieve the purpose of home care.

### 1. FIELD OF THE INVENTION

The present invention relates to a flexible biomonitor, and more particularly to a flexible biomonitor with high-density flexible substrate that adopts radio frequency identification (RFID) and micro-sensing technologies.

### 2. BACKGROUND OF THE INVENTION

According to statistic data of Frost and Sullivan business consulting firm in Year 2000, America, Western Europe, and Asia hold almost 70 percent of global medical device market, wherein Asia holds about 17 percent of global medical device market. In the mean time, the report produced by Industrial Development Bureau, Ministry of Economic Affairs, R.O.C., indicates that the growth rate of yield of global medical device from 1992 to 2002 year is 14.83%. Moreover, the growth rate of Taiwanese medical care production value is even up to 18.5%. From the facts that Taiwanese has about one half the yearly income of American while Taiwan only has one-tenth the population of America and the production value of American remote home care market is USD 40 to 70 billion, The production value of Taiwanese remote home care market is estimated to be USD 2 to 3.5 billion per year. Accordingly, the medical industry using wireless communication technology will substantially affect the future economy of Taiwan. It is expected that the developed plaster-type wireless transmission/reception module and key components thereof can assist the domestic wireless and biomedical manufacturers in mastering the business opportunity and equipping with favorable conditions and leading role for contending with overseas manufacturers.

In the meantime, according to the statistic, the marketing scale of the global medical device in the Year 2005 is estimated to be USD 192 billion with an average growth rate of 5%. The US Department of Commerce also estimates that ten newly risen markets including The Association of Southeast Asian Nations, The Chinese Economic Area (China, Taiwan, Hong Kong), South Korea, India, South Africa, Poland, Turkey, Mexico, Brazil, and Argentina will be formed in the year 2010. The amount of the medical devices imported by these ten newly risen markets will be multiplied, and the growth rate of the medical care expense of these markets will be two or three times over the developed countries. Asia area and Eastern Europe among them will have the rapidest growth rate. Regarding the domestic market of Taiwan, the medical care expense is also raising continually since Taiwanese had gradually pay more attention to the health and medical care and the Government sets the National Health Insurance Program into action. The market values of Taiwan in the years 1998 and 2000 are NTD 16.921 and 22.3 billion, respectively, and is estimated to be NTD 43 billion in the year 2005 with an average growth rate of 13.9%.

In order to enable the people with chronic disease and/or the people who require media to be able to move freely with the application preventive medicine for reducing disease and disability, the core technologies of information and communication industries are utilized to broaden the scope of the cared objects and meet the need of personalized long-term care via the borderless network such that the number of hospital visit can be reduced, and the probability of having nosocomial infection is also reduced. Accordingly, the lives of the aged persons and the person who require care become more plentiful. In order to achieve the purpose of remote home care, the real-time wireless monitoring module using the network will be the most human solution. In the mean time, with the solid foundation of wireless-related industries in Taiwan and the characteristic of short developing time required for developing the new electric medical device, it is the best time for developing the remote home care module and device. The product of the present invention is disclosed in accordance with this trend.

A physiological plaster having the wireless monitoring function is disclosed in a patent WO03065926, entitled “Wearable Biomonitor with Flexible Thinned Integrated Circuit”. In this cited patent, the plaster module is a single-sided plaster module. In other words, the sensor and the electronic device are mounted on the same side of the physiological plaster, which will cause the plaster to have the following drawbacks: (1) the area of the physiological plaster cannot be reduced; (2) the wear is less comfortable because of perspired sweat from the body; and (3) the electronic device cannot be properly protected.

### SUMMARY OF THE INVENTION

The primary object of the invention is to provide a flexible biomonitor that is a micro-system integrated with a flexible substrate, wherein the micro-system integrates a wireless transmission tag with a physiological sensor to form a plaster-sized module capable of attaching to the skin of a tested area for sensing and monitoring physiological signals.

Another object of the present invention is to provide a flexible biomonitor, in which a plurality of through holes for conducting electricity are formed on the flexible substrate enabling the electronic device and the sensor to be mounted respectively on the two sides of the flexible substrate for reducing occupied area and providing comfortable wear.

A further object of the present invention is to provide a flexible biomonitor capable of transmitting the physiological signals monitored thereby wirelessly using RFID technology. Accordingly, the purpose of home care is achieved by use of the network system of the medical center to perform the personalized remote real-time monitoring and to reduce the probability of having nosocomial infection by reducing the number of hospital visits of patients, aged persons, and children. Thereupon the medical quality is improved.

In order to achieve the aforementioned objects, the present invention provides a flexible biomonitor comprising a flexible substrate, a first circuit layer, a second circuit layer, a RFID sensing chip, a micro-antenna, and a power supply. Wherein, the flexible substrate, which can be made of a material selected from a group consisting of polyimide (PI), polyvinyl chloride (PVC), and polyvinyl alcohol (PVA) further comprises a plurality of through holes, each penetrating the flexible substrate and being filled with a conducting material; the first circuit layer connects to one side of the flexible substrate and further comprises a circuit layout and a plurality of gauges, such as sensing electrodes, mounted thereon for sensing a physiological phenomenon to generate a signal, the physiological phenomenon including heartbeat frequency, body temperature, and so on; the second circuit layer is connected to the other side of the flexible substrate opposite to the first circuit layer and is electrically connected with the first circuit layer through the plural through holes so as to receive the signal, and the second circuit layer further comprises a circuit layout and a plurality of integrated circuit (IC) devices mounted thereon; the RFID sensing chip is electrically connected with the second circuit layer, and operates cooperatively with the IC devices to perform a specific process on the signal; the micro-antenna is electrically connected with the second circuit layer for transmitting the processed signal in a wireless manner; and the power supply, which can be a battery in a preferred embodiment, is electrically connected with the second circuit layer for providing electricity for the first circuit layer, the second circuit layer, the RFID sensing chip, and the micro-antenna.

In a preferred embodiment of the invention, the flexible biomonitor further comprises a package covering the flexible substrate, the first circuit layer, the second circuit layer, the RFID sensing chip, the micro-antenna, and the power supply so as to protect the flexible biomonitor from moisture and dust pollution and simultaneously to provide a better feeling of skin contact. In addition, the package is made of a material selected from a group consisting of polydimethylsiloxane (PDMS), polyurethane (PU), and epoxy

Preferably, the special process includes signal amplifying, signal filtering, analog/digital signal converting, signal encoding, and signal decoding.

Preferably, the signal is received by a wireless reader.

Other aspects and advantages of the present invention will become apparent from the following detailed description, taken in conjunction with the accompanying drawings, illustrating by way of example the principles of the present invention.

### BRIEF DESCRIPTION OF THE DRAWINGS

FIG. 1 is a top view showing a flexible biomonitor in accordance with a preferred embodiment of the present invention;

FIG. 2 is a side view showing the flexible biomonitor in accordance with the preferred embodiment of the present invention; and

FIG. 3 is a schematic view showing the flexible biomonitor in accordance with the preferred embodiment of the present invention.

### DESCRIPTION OF THE PREFERRED EMBODIMENT

The above-mentioned features and advantages of this invention, and the manner of attaining them, will become more apparent and the invention will be better understood by reference to the following description of embodiments of the invention taken in conjunction with the drawings. But, the scope of the present invention is not limited to the drawings.

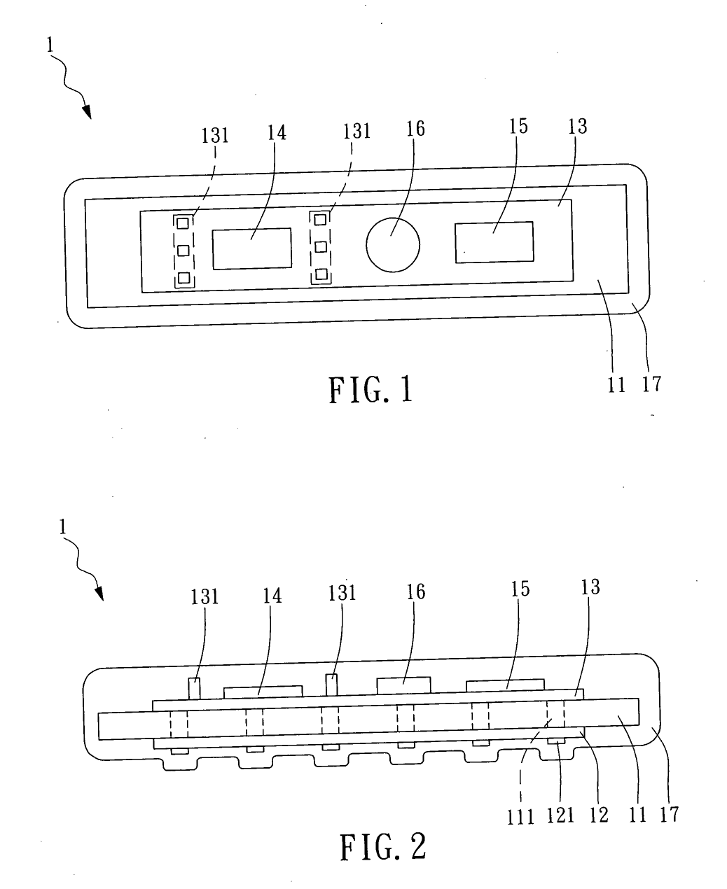

Referring to FIG. 1 and FIG. 2, a top view and a side view of a flexible biomonitor of a preferred embodiment of the present invention are shown, respectively. The flexible biomonitor 1 comprises a flexible substrate 11, a first circuit layer 12, a second circuit layer 13, a RFID sensing chip 14, a micro-antenna 15, a power supply 16, and a package 17.

The flexible substrate 11 comprises several through holes 111 formed thereon, wherein the through holes 111 are formed by the through hole manufacture process of the circuit board and mounted to penetrate through the flexible substrate 11. These through holes 111 are filled with a conducting material for providing electrical connection.

The first circuit layer 12 is connected to one side of the flexible substrate 11, wherein the first circuit layer 12 shown in the FIG. 2 is coupled with the lower portion of the flexible substrate 11. The first circuit layer 12 further comprises a circuit layout (not shown) and a plurality of gauges 121 mounted thereon. These gauges 121 are designed to sense a physiological phenomenon of the human body for generating a signal (not shown).

The second circuit layer 13 is connected to the other side of the flexible substrate 11, wherein the second circuit layer 13 shown in the FIG. 2 is coupled with the upper portion of the flexible substrate 11 and electrically connected with the first circuit layer 12 to transmit the signal. The second circuit layer 13 further comprises a circuit layout (not shown) and a plurality of IC devices 131 mounted thereon.

The RFID sensing chip 14 is electrically connected with the second circuit layer 13 and operated together with these IC devices 131 to perform a special treatment on the signal. The special treatment is signal amplifying, signal filtering, analog/digital signal conversion, signal encoding, signal decoding, etc.

The micro-antenna 15 is electrically connected with the second circuit layer 13 and designed to transmit the treated signal to a RFID receiver (not shown). Thereafter, the RFID receiver further transmits the signal to a receiving terminal, which is, for example, a short-distance sanatorium or a long-distance medical station (not shown), for real-time monitoring the physiological phenomenon.

The power supply 16 is electrically connected with the second circuit layer 13 for providing the electric power for the first circuit layer 12, the second circuit layer 13, the RFID sensing chip 14, and the micro-antenna 15.

The package 17 covers the flexible substrate 11, the first circuit layer 12, the second circuit layer 13, the RFID sensing chip 14, the micro-antenna 15, and the power supply 16 to protect the flexible biomonitor 1 from moisture and dust pollution and simultaneously to provide a better feel when the flexible biomonitor 1 touches the skin (not shown).

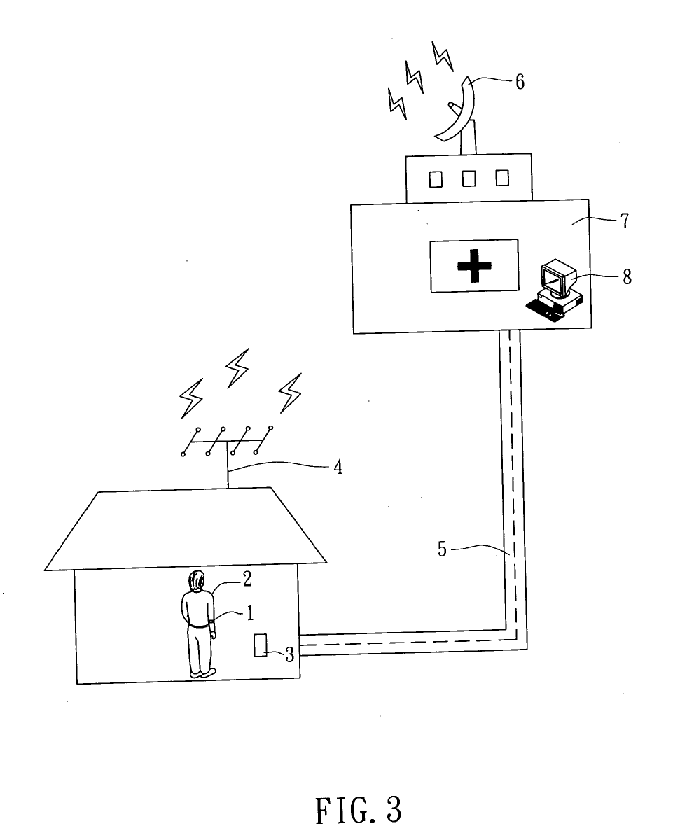

FIG. 3 is a schematic view showing the flexible biomonitor in accordance with the preferred embodiment of the present invention. As shown in FIG. 3, the user (patient) 2 wears the flexible biomonitor 1 of the present invention. When the flexible biomonitor 1 obtains a physiological phenomenon (not shown) of the user 2, it converts the physiological phenomenon into a signal carrier wave (not shown) and transmits the signal carrier wave to the RFID receiver 3 mounted in the user's house. Next, the RFID receiver 3 further transmits the signal carrier wave to a carrier wave receiver 6 mounted in a medical station 7. Thereafter, the carrier wave receiver 6 converts the signal carrier wave into digital data, which is shown on a monitor 8 by image. Alternatively, the RFID receiver 3 converts the signal carrier wave into a general digital signal (not shown), and then the digital signal is transmitted to the monitor 8 of the medical station 7 via the Ethernet 5 for showing the digital signal by image. Accordingly, the purpose of remote monitoring is achieved.

In the present invention, these circuit layers can be electrically connected by use of the copper conducting wires applied to the traditional surface or the through holes penetrated through the flexible substrate. Thereupon the occupied area of the flexible substrate is efficiently saved and the size of the flexible biomonitor is reduced. In the meantime, the IC devices are all kinds of active and passive IC devices capable of amplifying signal, filtering signal, converting analog/digital signal, encoding signal, decoding signal, etc. These gauges are sensing electrodes.

Besides, the power supply disclosed in this preferred embodiment of the present invention is a flexible battery, and the monitored physiological signal is the user's heartbeat frequency, body temperature, etc. The flexible substrate is made of a material such as polyimide (PI), polyvinyl chloride (PVC), polyvinyl alcohol (PVA), etc. The package is made of a material such as polydimethylsiloxane (PDMS), polyurethane (PU), epoxy, etc.

In the present invention, the package can completely cover the flexible substrate, the first circuit layer, the second circuit layer, the RFID sensing chip, the micro-antenna, and the power supply. Nevertheless, when these gauges are sensing electrodes that need to touch the user's skin directly, the package covers the flexible substrate, the first circuit layer, the second circuit layer, the RFID sensing chip, the micro-antenna, and the power supply and exposes these gauges. Accordingly, these gauges are allowed to touch the user's skin directly.

While the preferred embodiment of the invention has been set forth for the purpose of disclosure, modifications of the disclosed embodiment of the invention as well as other embodiments thereof may occur to those skilled in the art. Accordingly, the appended claims are intended to cover all embodiments which do not depart from the spirit and scope of the invention.

What is claimed is:

1. A flexible biomonitor, comprising:
a flexible substrate;
a circuit apparatus, connected with the flexible substrate, the circuit apparatus having a circuit layout mounted thereon and further comprising:
a first circuit layer, connected with one side of the flexible substrate;
a second circuit layer connected with the other side of the flexible substrate, and being electrically connected with the first circuit layer; and
a plurality of gauges, each capable of sensing a physiological phenomenon to generate a signal;
a radio frequency identification (RFID) sensing chip, electrically connected with the circuit apparatus for operating cooperatively with the circuit apparatus to perform a process on the signal;
a micro-antenna electrically connected with the circuit apparatus for transmitting the signal in a wireless manner; and
a power supply electrically connected with the circuit apparatus for providing electric power for the circuit apparatus, the RFID sensing chip, and the micro-antenna.

2. The flexible biomonitor of claim 1, further comprising a package, covering the flexible substrate, the circuit apparatus, the RFID sensing chip, the micro-antenna, and the power supply.

3. The flexible biomonitor of claim 2, wherein the package is made of a material selected from a group consisting of polydimethylsiloxane (PDMS), polyurethane (PU), and epoxy.

4. The flexible biomonitor of claim 1, wherein the flexible substrate further comprises a plurality of through holes, each penetrating therethrough and filled with a conducting material.

5. The flexible biomonitor of claim 4, wherein the first circuit layer is electrically connected with the second circuit layer via the through holes.

6. The flexible biomonitor of claim 1, wherein the circuit apparatus further comprises a plurality of integrated circuit (IC) devices.

7. The flexible biomonitor of claim 6, wherein the IC devices are active IC devices.

8. The flexible biomonitor of claim 6, wherein the IC devices are passive IC devices.

9. The flexible biomonitor of claim 1, wherein the gauges are sensing electrodes.

10. The flexible biomonitor of claim 1, wherein the physiological phenomenon is heartbeat frequency.

11. The flexible biomonitor of claim 1, wherein the physiological phenomenon is body temperature.

12. The flexible biomonitor of claim 1, wherein the special process includes at least a procedure selected from the group consisting of signal amplifying, signal filtering, analog/digital signal converting, signal encoding, and signal decoding.

13. The flexible biomonitor of claim 1, wherein the power supply is a flexible battery.

14. The flexible biomonitor of claim 1, wherein the flexible substrate is made of a material selected from a group consisting of polyimide (PI), polyvinyl chloride (PVC), and polyvinyl alcohol (PVA).

15. The flexible biomonitor of claim 1, wherein the signal is received by a wireless reader.
